(19) United States
(12) Patent Application Publication
(10) Pub. No.: US 2026/0014091 A1
(43) Pub. Date: Jan. 15, 2026

(54) COMPOSITIONS AND METHODS OF MAKING AND USE THEREOF

(71) Applicant: Ohio State Innovation Foundation, Columbus, OH (US)

(72) Inventors: Megan Allyn, Columbus, OH (US); Katelyn Reilly, Columbus, OH (US)

(21) Appl. No.: 19/262,468

(22) Filed: Jul. 8, 2025

Related U.S. Application Data
(60) Provisional application No. 63/668,946, filed on Jul. 9, 2024.

(51) Int. Cl. A61K 9/51; A61K 31/506; A61P 27/02

(52) U.S. Cl. CPC A61K 9/5146; A61K 31/506; A61P 27/02

(57) ABSTRACT

Disclosed herein are compositions and methods of making and use thereof. For example, disclosed herein are pharmaceutical compositions comprising: a plurality of redox-responsive disulfide nanoparticles; and a tyrosine kinase inhibitor encapsulated within each of the redox-responsive disulfide nanoparticles. Also disclosed herein are methods of treating or preventing an ocular injury, disease, or disorder in a subject in need thereof, the methods comprising administering a therapeutically effective amount of a plurality of redox-responsive disulfide nanoparticles and/or any of the pharmaceutical compositions disclosed herein to the subject.

### CROSS-REFERENCE TO RELATED APPLICATIONS

This application claims the benefit of priority to U.S. Provisional Application No. 63/668,946 filed Jul. 9, 2024, which is hereby incorporated herein by reference in its entirety.

### STATEMENT OF GOVERNMENT SUPPORT

This invention was made with government support under HT9425-23-1-0782 awarded by the US Army Medical Research and Development Command. The government has certain rights in the invention.

### BACKGROUND

Compositions with improved properties are needed. For example, improved drug delivery systems for prevention of vision loss are needed. The compositions and methods discussed herein address these and other needs.

### SUMMARY

In accordance with the purposes of the disclosed compositions and methods as embodied and broadly described herein, the disclosed subject matter relates to compositions and methods of making and use thereof.

For example, disclosed herein are pharmaceutical compositions comprising: a plurality of redox-responsive disulfide nanoparticles; and a tyrosine kinase inhibitor encapsulated within each of the redox-responsive disulfide nanoparticles.

In some examples, the redox-responsive disulfide nanoparticles are derived from a phospholipid-PEG. In some examples, the redox-responsive disulfide nanoparticles are derived from diacylphospholipid-poly(ethylene glycol). In some examples, the redox-responsive disulfide nanoparticles are derived from Thiol 1,2-Dioleoyl-sn-glycero-3-phosphoethanolamine (DOPE)-polyethylene glycol (PEG).

In some examples, the redox-responsive disulfide nanoparticles are derived from 1,2-distearoyl-sn-glycero-3-phosphoethanolamine-poly(ethylene glycol) and a thiol containing polymer precursor. In some examples, the thiol containing polymer precursor comprises 1,4-butane diol bis(thioglycolate).

In some examples, the redox-responsive disulfide nanoparticles are substantially spherical in shape.

In some examples, the redox-responsive disulfide nanoparticles have an average particle size of from 30 nanometers (nm) to 10 micrometers (microns, μm). In some examples, the redox-responsive disulfide nanoparticles have an average particle size of from 30 nm to 950 nm.

In some examples, the redox-responsive disulfide nanoparticles have an average particle size of from 90 nm to 7 μm. In some examples, the redox-responsive disulfide nanoparticles have an average particle size of from 100 nanometers (nm) to 500 nanometers. In some examples, the redox-responsive disulfide nanoparticles have an average particle size of from 200 to 250 nanometers, such as 227 nm.

In some examples, the redox-responsive disulfide nanoparticles have a polydispersity index of 1.0 or less. In some examples, the redox-responsive disulfide nanoparticles have a polydispersity index of 0.85 or less. In some examples, the redox-responsive disulfide nanoparticles have a polydispersity index of 0.5 or less. In some examples, the redox-responsive disulfide nanoparticles have a polydispersity index of 0.3 or less.

In some examples, the tyrosine kinase inhibitor is hydrophobic.

In some examples, the tyrosine kinase inhibitor comprises dasatinib.

In some examples, the tyrosine kinase inhibitor is encapsulated within the redox-responsive disulfide nanoparticle with an encapsulation efficiency of 50% or more, such as 80% or more.

In some examples, the redox-responsive disulfide nanoparticles encapsulating the tyrosine kinase inhibitor has an average Zeta potential of from −20 to −40 mV. In some examples, the redox-responsive disulfide nanoparticles encapsulating the tyrosine kinase inhibitor can have an average Zeta potential of from −25 to −35 mV. In some examples, the redox-responsive disulfide nanoparticles encapsulating the tyrosine kinase inhibitor has an average Zeta potential of from −25 to −30 mV. In some examples, the redox-responsive disulfide nanoparticles encapsulating the tyrosine kinase inhibitor has an average Zeta potential of −29.2±1.0 mV. In some examples, the redox-responsive disulfide nanoparticles encapsulating the tyrosine kinase inhibitor has an average Zeta potential of −25.5±1.0 mV.

In some examples, the pharmaceutical composition releases the tyrosine kinase inhibitor in response to a redox condition, for example when subjected to oxidative stress and/or in the presence of reactive oxygen species.

In some examples, the pharmaceutical composition is substantially non-toxic at concentration of 500 μg/mL or less. In some examples, the pharmaceutical composition is substantially non-toxic at concentration of 200 μg/mL or less. In some examples, the pharmaceutical composition is substantially non-toxic at concentration of 100 μg/mL or less.

In some examples, the pharmaceutical composition further comprises a solvent, a carrier, an excipient, an additional therapeutic agent (optionally encapsulated within the redox-responsive disulfide nanoparticles), or a combination thereof.

Also disclosed herein are methods of making any of the pharmaceutical compositions disclosed herein. In some examples, the method comprises oil-in-water self-assembly.

Also disclosed herein are methods of treating or preventing an ocular injury, disease, or disorder in a subject in need thereof, the methods comprising administering a therapeutically effective amount of a plurality of redox-responsive disulfide nanoparticles to the subject.

In some examples, the redox-responsive disulfide nanoparticles are derived from a phospholipid-PEG. In some examples, the redox-responsive disulfide nanoparticles are derived from diacylphospholipid-poly(ethylene glycol). In some examples, the redox-responsive disulfide nanoparticles are derived from Thiol 1,2-Dioleoyl-sn-glycero-3-phosphoethanolamine (DOPE)-polyethylene glycol (PEG).

In some examples, the redox-responsive disulfide nanoparticles are derived from 1,2-distearoyl-sn-glycero-3-phosphoethanolamine-poly(ethylene glycol) and a thiol containing polymer precursor. In some examples, the thiol containing polymer precursor comprises 1,4-butane diol bis(thioglycolate).

In some examples, the redox-responsive disulfide nanoparticles are substantially spherical in shape.

In some examples, the redox-responsive disulfide nanoparticles have an average particle size of from 30 nanometers (nm) to 10 micrometers (microns, μm). In some examples, the redox-responsive disulfide nanoparticles have an average particle size of from 30 nm to 950 nm.

In some examples, the redox-responsive disulfide nanoparticles have an average particle size of from 90 nm to 7 μm. In some examples, the redox-responsive disulfide nanoparticles have an average particle size of from 100 nanometers (nm) to 500 nanometers. In some examples, the redox-responsive disulfide nanoparticles have an average particle size of from 200 to 250 nanometers, such as 227 nm.

In some examples, the redox-responsive disulfide nanoparticles have a polydispersity index of 1.0 or less. In some examples, the redox-responsive disulfide nanoparticles have a polydispersity index of 0.85 or less. In some examples, the redox-responsive disulfide nanoparticles have a polydispersity index of 0.5 or less. In some examples, the redox-responsive disulfide nanoparticles have a polydispersity index of such as 0.3 or less.

In some examples, a therapeutic agent is encapsulated within each the redox-responsive disulfide nanoparticles.

In some examples, the therapeutic agent comprises an anticancer agent, an anti-inflammatory agent, an antimicrobial agent, an anti-oxidant agent, an antibody, a protein, or a combination thereof.

In some examples, the therapeutic agent comprises a chemotherapeutic agent, an immunotherapeutic agent, or a combination thereof.

In some examples, the therapeutic agent is hydrophobic.

In some examples, the therapeutic agent is a tyrosine kinase inhibitor.

In some examples, the tyrosine kinase inhibitor comprises dasatinib.

In some examples, the therapeutic agent is encapsulated within the redox-responsive disulfide nanoparticles with an encapsulation efficiency of 50% or more, such as 80% or more.

In some examples, the redox-responsive disulfide nanoparticles release the therapeutic agent in response to a redox condition, for example when subjected to oxidative stress and/or in the presence of reactive oxygen species.

Also disclosed herein are methods of treating or preventing an ocular injury, disease, or disorder in a subject in need thereof, the method comprising administering a therapeutically effective amount of any of the pharmaceutical compositions disclosed herein to the subject.

In some examples, the pharmaceutical composition releases the tyrosine kinase inhibitor in response to a redox condition, for example when subjected to oxidative stress and/or in the presence of reactive oxygen species.

In some examples, the ocular injury, disease, or disorder generates oxidative stress.

In some examples, the ocular injury, disease, or disorder generates reactive oxygen species (ROS).

In some examples, the ocular injury comprises injury to the cornea and/or ocular nerve.

In some examples, the ocular disease or disorder comprises a degenerative disease or disorder.

In some examples, the ocular disease or disorder comprises a retinal condition.

In some examples, the ocular disease or disorder comprises age-related macular degeneration, proliferative vitreoretinopathy (PVR), diabetic retinopathy, glaucoma, retinitis pigmentosa, inherited retinal diseases, retinal tears/holes/detachments, or a combination thereof.

In some examples, the ocular disease or disorder comprises age-related macular degeneration, proliferative vitreoretinopathy (PVR), or a combination thereof.

In some examples, the ocular disease or disorder comprises proliferative vitreoretinopathy (PVR).

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Dasatinib in vitro release.

FIG. 2. Schematic diagram of the preparation of the thiol-based redox-responsive nanoparticles and results.

FIG. 3. Zeta potential of Das-loaded nanoparticles.

FIG. 4. Diameter of thiol-based redox-responsive nanoparticles.

FIG. 5. Cell viability at different nanoparticle concentrations.

FIG. 6. Oxidative stress under different conditions.

FIG. 7. Cell viability at different nanoparticle concentrations.

FIG. 8. Panel A) Retinal detachment (RD) can require vitrectomy, or surgical removal of the vitreous and use of a tamponade to reattach the retina. Panel B) Complications include failed retinal reattachment and PVR-fibrosis and traction on the retina. Panel C) proposed herein is the development of a nanoencapsulated dasatinib for PVR prevention.

FIG. 9. Representative images showing inhibitory effect of dasatinib on PVR related pathological changes in vivo. Panel A) Twice a week injection of dasatinib solution prevented traction retinal detachment (TRD) in a pig model of PVR. Panel B) Sustained delivery of dasatinib using a particulate system prevented formation of a full thickness retinal fold (RF), while non-encapsulated dasatinib injection was ineffective.

FIG. 10A. PDA NPs scavenge and reduce intracellular ROS in ARPE-19 as shown by reduced green fluorescence of oxidation-sensitive diacetyldichlorofluorescein (DCF) probe with increased NP concentration (n=3, p<0.05).

FIG. 10B. PDA NPs demonstrate stimuli-responsive release of therapeutics for 2-6 months. Release rate of model therapeutic bovine serum albumin (BSA) is accelerated by increasing concentration (0-10 mM) of oxidative stressor H₂O₂.

FIG. 11A. PDS NPs significantly scavenge and reduce ROS induced by 200 μM H₂O₂ in ARPE-19 at 20 ug/mL NP concentration (n=3, p<0.05).

FIG. 11B. PDS NPs demonstrate responsive release of Das with less than 10% released by day 10. Release rate of is accelerated by oxidative stress (n=3, p<0.05).

FIG. 12. TEM of example unloaded PDA NPs (scale bar=100 nm).

FIG. 13. HPLC method development to quantify dasatinib.

FIG. 14A-FIG. 14C. Compared to saline, 500 μg/mL PDA NPs demonstrate no significant differences in FIG. 14A) ERG oscillatory potentials (OP), FIG. 14B) ERG b-wave amplitudes, and FIG. 14C) retinal layer thickness (OCT) 14 days after intravitreal injection in chicks (n=9).

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps. As used in the specification and in the claims, the term “comprising” can include the aspects “consisting of” and “consisting essentially of.”

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

When the specific values are disclosed between two end values, it is understood that these end values can also be included.

For the terms “for example” and “such as,” and grammatical equivalences thereof, the phrase “and without limitation” is understood to follow unless explicitly stated otherwise. It is further understood that these phrases are not used in a restrictive sense, but for explanatory purposes. “Exemplary” means “an example of” and is not intended to convey an indication of a preferred or ideal embodiment.

Values can be expressed herein as an “average” value. “Average” generally refers to the statistical mean value.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “and/or” includes any and all combinations of one or more of the associated listed items.

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition.

The expressions “ambient temperature” and “room temperature” as used herein are understood in the art and refer generally to a temperature from about 20° C. to about 35° C.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight of component Y, components X and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

A volume percent (vol %) of a component, unless specifically stated to the contrary, is based on the total volume of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It is understood that the term “salt,” as used herein, refers to a chemical compound that can be formed form a reaction between an acid and a base. It is understood that the term “salt,” as used herein, encompasses both inorganic and organic salts capable of providing the desired properties to the composition. In still further aspects, a cation of the disclosed herein salts is a metal cation.

“Phase,” as used herein, generally refers to a region of a material having a substantially uniform composition which is a distinct and physically separate portion of a heterogeneous system. The term “phase” does not imply that the material making up a phase is a chemically pure substance, but merely that the chemical and/or physical properties of the material making up the phase are essentially uniform throughout the material, and that these chemical and/or physical properties differ significantly from the chemical and/or physical properties of another phase within the material. Examples of physical properties include density, thickness, aspect ratio, specific surface area, porosity, and dimensionality. Examples of chemical properties include chemical composition.

By “continuous” it is meant a phase such that all points within the phase are directly connected, so that for any two points within a continuous phase there exists a path which connects the two points and does not leave the phase.

As used herein, “molecular weight” refers to number average molecular weight as measured by ¹H NMR spectroscopy, unless indicated otherwise.

As used herein the term “plurality” means 2 or more (e.g., 3 or more; 4 or more; 5 or more; 10 or more; 15 or more; 20 or more; 25 or more; 30 or more; 40 or more; 50 or more; 75 or more; 100 or more; 150 or more; 200 or more; 250 or more; 300 or more; 400 or more; 500 or more; 750 or more; 1000 or more; 1500 or more; 2000 or more; 2500 or more; 3000 or more; 4000 or more; or 5000 or more).

As used herein, the term “encapsulation,” or grammatical equivalent, refers to the process of confining an individual compound or molecule within a nanoparticle.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), birds, and insects. “Subject” can also include a mammal, such as a primate or a human. Thus, the subject can be a human or veterinary patient. The term “patient” refers to a subject under the treatment of a clinician, e.g., physician.

As used herein, antimicrobials include, for example, antibacterials, antifungals, and antivirals. As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, “reduce” or other forms of the word, such as “reducing” or “reduction,” refers to lowering of an event or characteristic (e.g., microbe population/infection). It is understood that the reduction is typically in relation to some standard or expected value, in other words it is relative, but that it is not always necessary for the standard or relative value to be referred to. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of microbial infections, “treating,” “treat,” and “treatment” as used herein, refers to partially or completely inhibiting or reducing the microbial infections which the subject is suffering. In one embodiment, this term refers to an action that occurs while a patient is suffering from, or is diagnosed with, the microbial infections, which reduces the severity of the condition, or retards or slows the progression of the condition. Treatment need not result in a complete cure of the condition; partial inhibition or reduction of the microbial infections is encompassed by this term.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. In some examples, the term “nucleic acid” as used herein means natural and synthetic DNA, RNA, oligonucleotides, oligonucleosides, and derivatives thereof. For ease of discussion, such nucleic acids are at times collectively referred to herein as “constructs,” “plasmids,” or “vectors.”

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

While aspects of the present invention can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

Unless otherwise defined, all technical and scientific terms used herein have the same meaning as commonly understood by one of ordinary skill in the art to which this invention belongs.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety.

The prefix Cₙ-Cₘ preceding a group or moiety indicates, in each case, the possible number of carbon atoms in the group or moiety that follows. For example, the term “Cₙ-Cₘ” (or “Cₙ₋ₘ”) employed alone or in combination with other terms refers to a hydrocarbon group that may be straight-chain or branched, having n to m carbons. It is understood that the terms Cₙ₋ₘ and Cₙ-Cₘ can be used interchangeably and just to show that the specific compound has between n to m carbons.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

“Zwitterionic” or “zwitterion” as used herein refers to a neutral molecule with a positive (or cationic) and a negative (or anionic) electrical charge at different locations within the same molecule.

As used herein, the term “substituted” means that a hydrogen atom is removed and replaced by a substituent. It is contemplated to include all permissible substituents of organic compounds. As used herein, the phrase “optionally substituted” means unsubstituted or substituted. It is to be understood that substitution at a given atom is limited by valency. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. In still further aspects, it is understood that when the disclosure describes a group being substituted, it means that the group is substituted with one or more (i.e., 1, 2, 3, 4, or 5) groups as allowed by valence selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “compound,” as used herein, is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein can also include tautomeric forms. Tautomeric forms result from the swapping of a single bond with an adjacent double bond together with the concomitant migration of a proton. Tautomeric forms include prototropic tautomers, which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include hydrogen, tritium, and deuterium.

Also provided herein are salts of the compounds described herein. It is understood that the disclosed salts can refer to derivatives of the disclosed compounds wherein the parent compound is modified by converting an existing acid or base moiety to its salt form. Examples of the salts include, but are not limited to, mineral or organic acid salts of basic residues such as amines; alkali or organic salts of acidic residues such as carboxylic acids; and the like. The salts of the compounds provided herein include the conventional non-toxic salts of the parent compound formed, for example, from non-toxic inorganic or organic acids. The salts of the compounds provided herein can be synthesized from the parent compound that contains a basic or acidic moiety by conventional chemical methods. Generally, such salts can be prepared by reacting the free acid or base forms of these compounds with a stoichiometric amount of the appropriate base or acid in water or an organic solvent or in a mixture of the two. In various aspects, nonaqueous media like ether, ethyl acetate, alcohols (e.g., methanol, ethanol, isopropanol, or butanol), or acetonitrile (ACN) can be used.

As used herein, chemical structures that contain one or more stereocenters depicted with dashed and bold bonds are meant to indicate the absolute stereochemistry of the stereocenter(s) present in the chemical structure. As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible stereoisomeric forms of the compound (e.g., diastereomers and enantiomers) and mixtures thereof. Structures with a single bold or dashed line and at least one additional simple line encompass a single enantiomeric series of all possible diastereomers.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

A dash (“-”) that is not between two letters or symbols is used to indicate a point of attachment for a substituent. For example, —(C═O)NH₂ is attached through the carbon of the keto (C═O) group.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied. It is further understood that throughout the specification, “alkyl” can also be referred to as a linking group of saturated hydrocarbons that are divalent radicals. In other words, in a broader description, the term “alkyls” also encompasses alkylenes. It is further understood that the term “alkyl” covers saturated hydrocarbons that are multivalent radicals.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

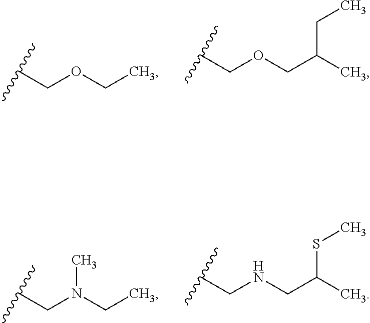

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include an aromatic carbocyclic group of from 3 to 50 carbon atoms. Aryl groups can include a single ring or multiple condensed rings. In some examples, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” as used herein are represented by the formula —NR¹R², where R¹ and R² can each be substitution groups as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

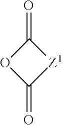

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

As used herein, the term “carbamyl” refers to a group of formula —C(O)NH₂.

A “carbonate ester” group as used herein is represented by the formula Z¹OC(O)OZ².

The term “cyano” as used herein is represented by the formula —CN.

The term “ester” as used herein is represented by the formula —OC(O)Z¹ or

—C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

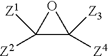

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein comprises the formula —S—.

The term “thiol” as used herein is represented by the formula —SH.

The term “sulfonylamino” or “sulfonamide,” as used herein, is represented by the formula —S(O)₂NH—.

In general, the inclusion of the prefix “alk” in front of a substituent name indicates there is an alkyl group (as defined herein) connecting the named substituent with the rest of the compound. For example, “alkaryl” (which is a subset of alkyl) refers to an alkyl group substituted by an aryl group, wherein the point of attachment is on the alkyl moiety and “alkheteroaryl” (which is a subset of “alkyl”) refers to an alkyl group substituted by a heteroaryl group, wherein the point of attachment is on the alkyl moiety. The number of carbon atoms may be specified in the alkyl chain, the named substituent, or both. For example, C₁₋₂alkC₆aryl refers to a phenyl ring (which may be substituted) connected via a 1-2 carbon alkylene group.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

“R¹,” “R²,” “R³,” “Rⁿ”, etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amino group, an alkyl group, a halide, and the like. Depending upon the groups that are selected, a first group can be incorporated within a second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As used herein, the designation of a polyvalent moiety without specifying the specific order of attachment is intended to cover all possible arrangements. By way of example, a compound that is represented by the formula:

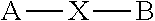

wherein X is NHC(═O) embraces both

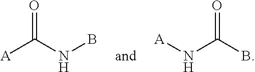

As used herein, a chemical bond depicted: [symbol] represents either a single, double, or triple bond, valency permitting. By way of example,

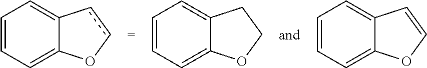

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}, C(O)OR, and C(O)NR₂ (wherein R is H or an alkyl, aryl, heteroaryl, cycloalkyl, heterocyclyl group) as well as alkyl group substituted with one or more of those group.

An electron-donating group is a functional group or atom that pushes electron density away from itself towards other portions of the molecule, e.g., through resonance and/or inductive effects. Exemplary electron-donating groups include unsubstituted alkyl or aryl groups, OR and N(R)₂, and alkyl groups substituted with one or more OR and N(R)₂ groups.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Pharmaceutical Compositions

Disclosed herein are compositions and methods of making and use thereof.

For example, disclosed herein are redox-responsive disulfide nanoparticles for therapeutic delivery.

For example, disclosed herein are redox-responsive nanoparticle systems for tyrosine kinase inhibitor delivery to treat retinal conditions, such as macular degeneration and proliferative vitreoretinopathy (PVR).

In some examples, the compositions comprise a particle.

As used herein, “a particle” and “the particle” are meant to include any number of particles. Thus, for example “a particle” includes one or more particles. In some embodiments, the particle can comprise a plurality of particles.

For example, disclosed herein are pharmaceutical compositions comprising: a plurality of redox-responsive disulfide nanoparticles; and a tyrosine kinase inhibitor encapsulated within each of the redox-responsive disulfide nanoparticles.

In some examples, the redox-responsive disulfide nanoparticles are derived from a phospholipid-PEG. In some examples, the redox-responsive disulfide nanoparticles are derived from diacylphospholipid-poly(ethylene glycol). In some examples, the redox-responsive disulfide nanoparticles are derived from Thiol 1,2-Dioleoyl-sn-glycero-3-phosphoethanolamine (DOPE)-polyethylene glycol (PEG).

In some examples, the redox-responsive disulfide nanoparticles are derived from 1,2-distearoyl-sn-glycero-3-phosphoethanolamine-poly(ethylene glycol) and a thiol containing polymer precursor. In some examples, the thiol containing polymer precursor comprises 1,4-butane diol bis(thioglycolate).

The plurality of redox-responsive disulfide nanoparticles can comprise particles of any shape, such as a polyhedron (e.g., a platonic solid, a prism, a pyramid), a cylinder, a hemicylinder, an elliptical cylinder, a hemi-elliptical cylinder, a sphere, a hemisphere, a cone, a semicone, etc. In some examples, the plurality of redox-responsive disulfide nanoparticles can have a regular shape, an irregular shape, an isotropic shape, an anisotropic shape, or a combination thereof. In some examples, the plurality of redox-responsive disulfide nanoparticles can have an isotropic shape or an anisotropic shape. In some examples, the plurality of redox-responsive disulfide nanoparticles can have a shape that is substantially spherical.

The plurality of redox-responsive disulfide nanoparticles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The plurality of redox-responsive disulfide nanoparticles can, for example, have an average particle size of 30 nanometers (nm) or more (e.g., 35 nm or more, 40 nm or more, 45 nm or more, 50 nm or more, 60 nm or more, 70 nm or more, 80 nm or more, 90 nm or more, 100 nm or more, 125 nm or more, 150 nm or more, 175 nm or more, 200 nm or more, 225 nm or more, 250 nm or more, 300 nm or more, 350 nm or more, 400 nm or more, 450 nm or more, 500 nm or more, 600 nm or more, 700 nm or more, 800 nm or more, 900 nm or more, 1 micrometer (micron, μm) or more, 1.5 μm or more, 2 μm or more, 2.5 μm or more, 3 μm or more, 3.5 μm or more, 4 μm or more, 4.5 μm or more, 5 μm or more, 6 μm or more, 7 μm or more, 8 μm or more, or 9 μm or more). In some examples, the redox-responsive disulfide nanoparticles have an average particle size of 10 μm or less (e.g., 9 μm or less, 8 μm or less, 7 μm or less, 6 μm or less, 5 μm or less, 4.5 μm or less, 4 μm or less, 3.5 μm or less, 3 μm or less, 2.5 μm or less, 2 μm or less, 1.5 μm or less, 1 μm or less, 900 nanometers (nm) or less, 800 nm or less, 700 nm or less, 600 nm or less, 500 nm or less, 450 nm or less, 400 nm or less, 350 nm or less, 300 nm or less, 250 nm or less, 225 nm or less, 200 nm or less, 175 nm or less, 150 nm or less, 125 nm or less, 100 nm or less, 90 nm or less, 80 nm or less, 70 nm or less, 60 nm or less, 50 nm or less, 45 nm or less, 40 nm or less, or 35 nm or less). The average particle size of the redox-responsive disulfide nanoparticles can range from any of the minimum values described above to any of the maximum values described above. For example, the redox-responsive disulfide nanoparticles have an average particle size of from 30 nanometers (nm) to 10 micrometers (microns, μm) (e.g., from 30 nm to 1 μm, from 1 μm to 10 μm, from 30 nm to 300 nm, from 300 nm to 1 μm, from 1 μm to 5 μm, from 5 μm to 10 μm, from 30 nm to 5 μm, from 30 nm to 1 μm, from 30 nm to 950 nm, from 30 nm to 750 nm, from 30 nm to 500 nm, from 30 nm to 250 nm, from 50 nm to 10 μm, from 100 nm to 10 μm, from 100 nm to μm, from 250 nm to 10 μm, from 500 nm to 10 μm, from 750 nm to 10 μm, from 50 nm to 5 μm, from 90 nm to 7 μm, from 100 nm to 1 μm, or from 100 nm to 500 nm). In some examples, the redox-responsive disulfide nanoparticles have an average particle size of from 90 nm to 7 μm. In some examples, the redox-responsive disulfide nanoparticles have an average particle size of 30 nm to 950 nm. In some examples, the redox-responsive disulfide nanoparticles have an average particle size of from 100 nanometers (nm) to 500 nanometers. In some examples, the redox-responsive disulfide nanoparticles have an average particle size of from 200 to 250 nanometers, such as 227 nm.

With respect to particle size distribution characterization, a parameter used to define the size range of the plurality of redox-responsive disulfide nanoparticles is called the “polydispersity index” (PDI). The term “polydispersity” (or “dispersity” as recommended by IUPAC) is used to describe the degree of non-uniformity of a size distribution of particles. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In some examples, the redox-responsive disulfide nanoparticles the redox-responsive disulfide nanoparticles have a polydispersity index of 1.0 or less (e.g., 0.95 or less, 0.90 or less, 0.85 or less, 0.80 or less, 0.75 or less, 0.70 or less, 0.65 or less, 0.60 or less, 0.55 or less, 0.50 or less, 0.49 or less, 0.48 or less, 0.47 or less, 0.46 or less, 0.45 or less, 0.44 or less, 0.43 or less, 0.42 or less, 0.41 or less, 0.40 or less, 0.39 or less, 0.38 or less, 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the redox-responsive disulfide nanoparticles the redox-responsive disulfide nanoparticles have a polydispersity index of 0.85 or less (e.g., 0.80 or less, 0.75 or less, 0.70 or less, 0.65 or less, 0.60 or less, 0.55 or less, 0.50 or less, 0.49 or less, 0.48 or less, 0.47 or less, 0.46 or less, 0.45 or less, 0.44 or less, 0.43 or less, 0.42 or less, 0.41 or less, 0.40 or less, 0.39 or less, 0.38 or less, 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less)

In some examples, the redox-responsive disulfide nanoparticles the redox-responsive disulfide nanoparticles have a polydispersity index of 0.5 or less (e.g., 0.49 or less, 0.48 or less, 0.47 or less, 0.46 or less, 0.45 or less, 0.44 or less, 0.43 or less, 0.42 or less, 0.41 or less, 0.40 or less, 0.39 or less, 0.38 or less, 0.37 or less, 0.36 or less, 0.35 or less, 0.34 or less, 0.33 or less, 0.32 or less, 0.31 or less, 0.30 or less, 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the redox-responsive disulfide nanoparticles the redox-responsive disulfide nanoparticles have a polydispersity index of 0.3 or less (e.g., 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some example, the plurality of redox-responsive disulfide nanoparticles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

The tyrosine kinase inhibitor can comprise any suitable tyrosine kinase inhibitor consistent with the compositions and methods herein. Examples of tyrosine kinase inhibitors include, but are not limited to acalabrutinib, adavosertib, afatinib, apatinib, axitinib, aumolertinib, avapritinib, avutometinib/defactinib, cabozantinib, canertinib, crenolanib, crizotinib, dabrafenib, damnacanthal, dasatinib, entospletinib, entrectinib, erlotinib, foretinib, fostamatinib, gefitinib, gilteritinib, glesatinib, HS-10365, ibrutinib, icotinib, imatinib, IDRX-42, lapatinib, lazertinib, linifanib, masitinib, mirdametinib, motesanib, mubritinib, nemtabrutinib, nilotinib, nintedanib, olverembatinib, pazopanib, pirtobrutinib, radotinib, remibrutinib, repotrectinib, ropsacitinib, savolitinib, sitravatinib, sorafenib, sunitinib, sunvozertinib, T790M, tesevatinib, tolebrutinib, trametinib, V600E, vandetanib, vatalanib, vemurafenib, zasocitinib, and combinations thereof. In some examples, the tyrosine kinase inhibitor comprises dasatinib.

In some examples, the tyrosine kinase inhibitor is hydrophobic.

In some examples, the tyrosine kinase inhibitor is encapsulated within the redox-responsive disulfide nanoparticle with an encapsulation efficiency of 50% or more (e.g., 55% or more, 60% or more, 65% or more, 70% or more, 75% or more, 80% or more, 85% or more, 90% or more, 95% or more, or 99% or more).

In some examples, the tyrosine kinase inhibitor is encapsulated within the redox-responsive disulfide nanoparticle with an encapsulation efficiency of 80% or more (e.g., 85% or more, 90% or more, 95% or more, or 99% or more).

In some examples, the redox-responsive disulfide nanoparticles encapsulating the tyrosine kinase inhibitor has an average Zeta potential of −20 mV or less (e.g., −21 mV or less, −22 mV or less, −23 mV or less, −24 mV or less, −25 mV or less, −26 mV or less, −27 mV or less, −28 mV or less, −29 mV or less, −30 mV or less, −31 mV or less, −32 mV or less, −33 mV or less, −34 mV or less, −35 mV or less, −36 mV or less, −37 mV or less, −38 mV or less, or −39 mV or less). In some examples, the redox-responsive disulfide nanoparticles encapsulating the tyrosine kinase inhibitor has an average Zeta potential of −40 mV or more (e.g., −39 mV or more, −38 mV or more, −37 mV or more, −36 mV or more, −35 mV or more, −34 mV or more, −33 mV or more, −32 mV or more, −31 mV or more, −30 mV or more, −29 mV or more, −28 mV or more, −27 mV or more, −26 mV or more, −25 mV or more, −24 mV or more, −23 mV or more, −22 mV or more, or −21 mV or more). The average Zeta potential of the redox-responsive disulfide nanoparticles encapsulating the tyrosine kinase inhibitor can range from any of the minimum values described above to any of the maximum values described above. For example, the redox-responsive disulfide nanoparticles encapsulating the tyrosine kinase inhibitor can have an average Zeta potential of from −20 to −40 mV (e.g., from −20 to −30 mV, from −30 to −40 mV, from −20 to −25 mV, from −25 to −30 mV, from −30 to −35 mV, from −35 to −40 mV, from −20 to −35 mV, from −25 to −40 mV, or from −25 to −35 mV). In some examples, the redox-responsive disulfide nanoparticles encapsulating the tyrosine kinase inhibitor can have an average Zeta potential of from −25 to −35 mV. In some examples, the redox-responsive disulfide nanoparticles encapsulating the tyrosine kinase inhibitor can have an average Zeta potential of from −25 to −30 mV. In some examples, the redox-responsive disulfide nanoparticles encapsulating the tyrosine kinase inhibitor can have an average Zeta potential of −29.2±1.0 mV. In some examples, the redox-responsive disulfide nanoparticles encapsulating the tyrosine kinase inhibitor can have an average Zeta potential of −25.5±1.0 mV.

In some examples, the pharmaceutical composition releases the tyrosine kinase inhibitor in response to a redox condition, for example when subjected to oxidative stress and/or in the presence of reactive oxygen species.

In some examples, the pharmaceutical composition is substantially non-toxic at concentration of 500 μg/mL or less (e.g., 450 μg/mL or less, 400 μg/mL or less, 350 μg/mL or less, 300 μg/mL or less, 250 μg/mL or less, 200 μg/mL or less, 150 μg/mL or less, 100 μg/mL or less, 75 μg/mL or less, 50 μg/mL or less, 25 μg/mL or less, or 10 μg/mL or less).

In some examples, the pharmaceutical composition is substantially non-toxic at concentration of 200 μg/mL or less (e.g., 150 μg/mL or less, 100 μg/mL or less, 75 μg/mL or less, 50 μg/mL or less, 25 μg/mL or less, or 10 μg/mL or less).

In some examples, the pharmaceutical composition is substantially non-toxic at concentration of 100 μg/mL or less (e.g., 75 μg/mL or less, 50 μg/mL or less, 25 μg/mL or less, or 10 μg/mL or less).

In some examples, the pharmaceutical composition further comprises a solvent, a carrier, an excipient, an additional therapeutic agent (optionally encapsulated within the redox-responsive disulfide nanoparticles), or a combination thereof.

In some examples, the pharmaceutical composition is administered to a subject. In some examples, the subject is a mammal. In some examples, the mammal is a primate. In some examples, the mammal is a human. In some examples, the human is a patient.

In some examples, the disclosed compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, intranasal, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

### Methods of Making

Also disclosed herein are methods of making any of the compositions disclosed herein. For example, also disclosed herein are methods of making any of the pharmaceutical compositions disclosed herein. In some examples, the method comprises oil-in-water self-assembly.

The compositions described herein can be prepared in a variety of ways known to one skilled in the art of organic synthesis or variations thereon as appreciated by those skilled in the art. The compositions described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compositions described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

### Methods of Use

Also disclosed herein are methods of use of any of the compositions disclosed herein.

For example also disclosed herein are methods of treating or preventing an ocular injury, disease, or disorder in a subject in need thereof, the method comprising administering a therapeutically effective amount of a plurality of redox-responsive disulfide nanoparticles to the subject.

In some examples, the plurality of redox-responsive disulfide nanoparticles can comprise any of those described herein above.

In some examples, a therapeutic agent is encapsulated within each the redox-responsive disulfide nanoparticles. In some examples, the therapeutic agent is hydrophobic.

The therapeutic agent can, for example, comprise an anticancer agent, an anti-inflammatory agent, an antimicrobial agent, an anti-oxidant agent, an antibody, a protein, or a combination thereof. As used herein, antimicrobials include, for example, antibacterials, antifungals, and antivirals.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper (I) cyanide, copper (II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2 (5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl) quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, ZMapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine. and combinations thereof.

In some examples, the therapeutic agent comprises an anticancer agent. In some examples, the therapeutic agent comprises a chemotherapeutic agent, an immunotherapeutic agent, or a combination thereof.

In some examples, the therapeutic agent can comprise a chemotherapeutic agent. Chemotherapy is the treatment of cancer with one or more cytotoxic anti-neoplastic drugs (e.g., chemotherapeutic agents) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapeutic agents include, but are not limited to, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C₂₂₅, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, Oprozomib, and combinations thereof. The anticancer agent can also include biopharmaceuticals such as, for example, antibodies.

Examples of suitable immunotherapeutic agents include, but are not limited to, alemtuzumab, cetuximab (ERBITUX), gemtuzumab, iodine 131 tositumomab, rituximab, tamoxifen, temozolomide, trastuzamab (HERCEPTIN), and combinations thereof.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent is a tyrosine kinase inhibitor. Examples of tyrosine kinase inhibitors include, but are not limited to acalabrutinib, adavosertib, afatinib, apatinib, axitinib, aumolertinib, avapritinib, avutometinib/defactinib, cabozantinib, canertinib, crenolanib, crizotinib, dabrafenib, damnacanthal, dasatinib, entospletinib, entrectinib, erlotinib, foretinib, fostamatinib, gefitinib, gilteritinib, glesatinib, HS-10365, ibrutinib, icotinib, imatinib, IDRX-42, lapatinib, lazertinib, linifanib, masitinib, mirdametinib, motesanib, mubritinib, nemtabrutinib, nilotinib, nintedanib, olverembatinib, pazopanib, pirtobrutinib, radotinib, remibrutinib, repotrectinib, ropsacitinib, savolitinib, sitravatinib, sorafenib, sunitinib, sunvozertinib, T790M, tesevatinib, tolebrutinib, trametinib, V600E, vandetanib, vatalanib, vemurafenib, and combinations thereof. In some examples, the tyrosine kinase inhibitor comprises dasatinib.

In some examples, the therapeutic agent is encapsulated within the redox-responsive disulfide nanoparticle with an encapsulation efficiency of 50% or more (e.g., 55% or more, 60% or more, 65% or more, 70% or more, 75% or more, 80% or more, 85% or more, 90% or more, 95% or more, or 99% or more).

In some examples, the therapeutic agent is encapsulated within the redox-responsive disulfide nanoparticle with an encapsulation efficiency of 80% or more (e.g., 85% or more, 90% or more, 95% or more, or 99% or more).

In some examples, the redox-responsive disulfide nanoparticles release the therapeutic agent in response to a redox condition, for example when subjected to oxidative stress and/or in the presence of reactive oxygen species.

Also disclosed herein are methods of treating or preventing an ocular injury, disease, or disorder in a subject in need thereof, the method comprising administering a therapeutically effective amount of any of the pharmaceutical compositions disclosed herein to the subject.

In some examples, the pharmaceutical composition releases the tyrosine kinase inhibitor in response to a redox condition, for example when subjected to oxidative stress and/or in the presence of reactive oxygen species.

In some examples, the subject is a mammal. In some examples, the mammal is a primate. In some examples, the mammal is a human. In some examples, the human is a patient.

In some examples, the ocular injury, disease, or disorder generates oxidative stress.

In some examples, the ocular injury, disease, or disorder generates reactive oxygen species (ROS).

In some examples, the ocular injury comprises injury to the cornea and/or ocular nerve.

In some examples, the ocular disease or disorder comprises a degenerative disease or disorder.

In some examples, the ocular disease or disorder comprises a retinal condition.

In some examples, the ocular disease or disorder comprises retinopathy of prematurity (ROP), retinoblastoma, uveal melanoma, age-related macular degeneration, proliferative vitreoretinopathy (PVR), diabetic retinopathy, glaucoma, retinitis pigmentosa, inherited retinal diseases, retinal tears/holes/detachments, or a combination thereof. In some examples, the ocular disease or disorder comprises age-related macular degeneration, proliferative vitreoretinopathy (PVR), diabetic retinopathy, glaucoma, retinitis pigmentosa, inherited retinal diseases, retinal tears/holes/detachments, or a combination thereof. In some examples, the ocular disease or disorder comprises age-related macular degeneration, proliferative vitreoretinopathy (PVR), or a combination thereof. In some examples, the ocular disease or disorder comprises proliferative vitreoretinopathy (PVR).

The methods of treatment of the disease or disorder described herein can further include treatment with one or more additional agents. The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

The specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

The methods, compounds, and compositions as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

In certain embodiments, it is desirable to target a nanoparticle using a targeting moiety that is specific to a cell type and/or tissue type. In some embodiments, a nanoparticle may be targeted to a particular cell, tissue, and/or organ using a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5): 439-62, 2003 and Abra et al., J. Liposome Res. 12:1-3, 2002.

### Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Redox-Responsive Disulfide Nanoparticles for Therapeutic Delivery

A redox-responsive nanoparticle delivery system was generated through oil-in-water self-assembly of 1,2-distearoyl-sn-glycero-3-phosphoethanolamine-poly(ethylene glycol)(DSPE-PEG) with a thiol containing polymer precursor based on published methods. A tyrosine kinase inhibitor (TKI) Dasatinib (Das) was dissolved in the oil (non-aqueous) phase before dropwise addition of the oil phase into ultrapure water under vigorous stirring to prepare nanoparticles. Benefits include encapsulation of hydrophobic therapeutics (e.g. Das) and redox-responsive release (higher release in presence of something like glutathione). This has applications in several areas, such as ocular drug delivery-particular applications include injury (cornea to optic nerve) or degenerative diseases.

This is the first description of polydisulfide like nanoparticles for ocular use. This is also the first encapsulation of tyrosine kinase inhibitors (for any application).

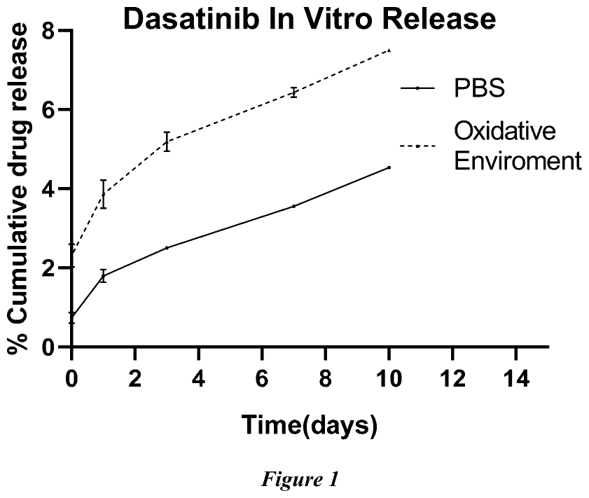

Dasatinib in vitro release is shown in FIG. 1.

### Example 2—Redox-Responsive Nanoparticle System for Tyrosine Kinase Inhibitor Delivery to Treat Retinal Conditions

Introduction: Reactive oxygen species (ROS) are implicated in many ocular conditions, including age-related macular degeneration and proliferative vitreoretinopathy (PVR). PVR development after retinal detachment stems from the sustained loss of cell-to-cell connection which results in elevated ROS, epithelial cell migration, and fibrotic membrane production on the retina, distorting or blocking vision. An objective of this study was to develop a redox-responsive nanoparticle delivery system for sustained release of a tyrosine kinase inhibitor (TKI), Dasatinib (Das), which has demonstrated potential to prevent PVR. An objective of this study was to investigate the redox-responsive delivery system for treatment of retinal conditions, particularly PVR. An objective of this study was to examine characteristics of a Dasatinib-loaded nanoparticle system.

Methods: The redox-responsive nanoparticle delivery system was generated through oil-in-water self-assembly of 1,2-distearoyl-sn-glycero-3-phosphoethanolamine-poly(ethylene glycol)₃₄₀₀ (DSPE-PEG_{3.4k}) with a thiol containing polymer precursor based on published methods. Das was dissolved in the oil (non-aqueous) phase before dropwise addition of the oil phase into ultrapure water under vigorous stirring. Particles were centrifuged and lyophilized for further characterization. Nanoparticle size and zeta potential were determined by Brookhaven ZetaPals analyzer. Drug loading and release profile were determined by UV-Vis spectroscopy at 323 nm. In vitro release of Das from particles was determined in sink conditions in phosphate buffered saline (PBS) or PBS supplemented with 10 mM glutathione for reducing conditions.

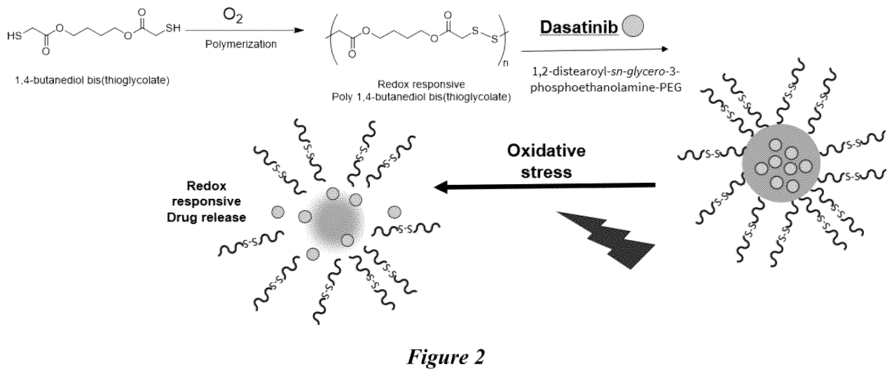

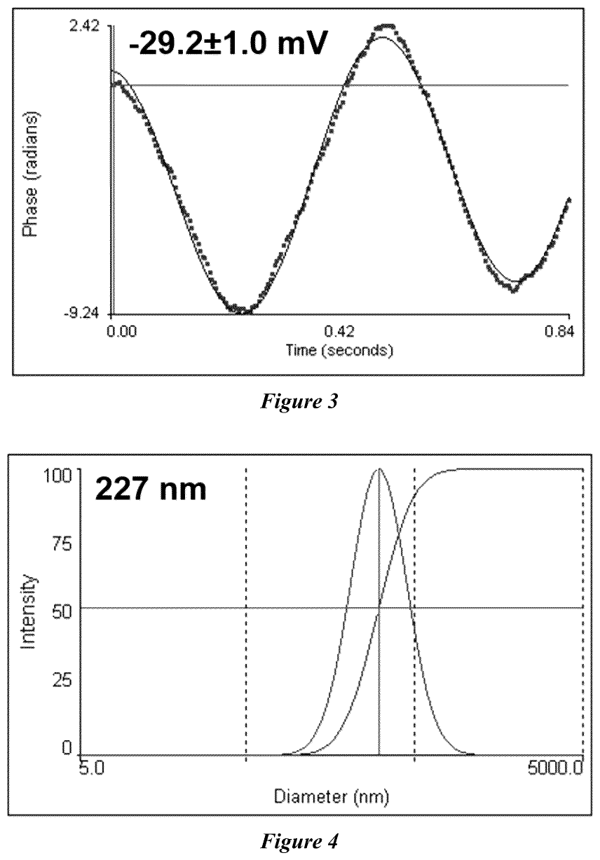

Results: Thiol-based redox-responsive nanoparticles were successfully prepared with 227 nm average diameter (FIG. 4) with 0.3 PDI. Zeta potential of Das-loaded nanoparticles was measured as −29.2±1.0 mV (FIG. 3). The drug entrapment efficiency of Das was greater than 80%. Release of Das from the nanoparticles in PBS demonstrated approximately 10% cumulative drug release after 24 hours (FIG. 1). Redox-responsive release of the therapeutic was also demonstrated after incubation with physiologically relevant levels of glutathione. Release rates increased in the presence of glutathione, with significant burst release. A schematic of the prepared particles and results are shown in FIG. 1 and FIG. 2.

Additional results are summarized in Table 1-Table 5. The total drug loaded for release was 1600 micrograms.

Conclusions/Impact: This is the first evaluation of this redox-responsive nanoparticle delivery system for release of a TKI for ocular applications. Thiol-based responsive nanoparticles were successfully prepared and loaded with Das with high entrapment efficiency and demonstrated higher release rates in the presence of glutathione. Ongoing studies include cellular uptake in primary retinal cells, establishing sustained release profiles with additional reducing agents, and evaluating prevention of fibrosis.

TABLE 1
HPLC Area.

| | Time point | Area R | Area S | Area V | Area W |
|---|---|---|---|---|---|
| Day 0 | 1 | 2000 | 2580.8 | 6557.7 | 7818.7 |
| Day 1 | 2 | 3245.3 | 3385.7 | 4684.7 | 5000 |
| Day 3 | 3 | 2500 | 1900 | 4405.7 | 3876.1 |
| Day 7 | 4 | 3264.8 | 3277.3 | 4144.6 | 3627 |
| Day 10 | 5 | 3108.3 | 3000.3 | 3571.1 | 3070.1 |

TABLE 2
Concentration (ppm).

| | Time point | R | S | V | W |
|---|---|---|---|---|---|
| Day 0 | 1 | 10.27007 | 13.25281 | 33.6765 | 40.15247 |
| Day 1 | 2 | 16.66541 | 17.38644 | 24.05756 | 25.67681 |
| Day 3 | 3 | 12.83786 | 9.756512 | 22.62473 | 19.90493 |
| Day 7 | 4 | 16.76555 | 16.82975 | 21.28383 | 18.62566 |
| Day 10 | 5 | 15.96183 | 15.40719 | 18.33858 | 15.76565 |

TABLE 3
Cumulative Drug release (ppm).

| | Time point | R | S | V | W |
|---|---|---|---|---|---|
| Day 0 | 1 | 10.27007 | 13.25281 | 33.6765 | 40.15247 |
| Day 1 | 2 | 26.93548 | 30.63926 | 57.73406 | 65.82927 |
| Day 3 | 3 | 39.77334 | 40.39577 | 80.35879 | 85.7342 |
| Day 7 | 4 | 56.53889 | 57.22552 | 101.6426 | 104.3599 |
| Day 10 | 5 | 72.50072 | 72.63271 | 119.9812 | 120.1255 |

TABLE 4
Percent cumulative drug released (ppm).

| | Time point | R | S | V | W |
|---|---|---|---|---|---|
| Day 0 | 1 | 0.641879 | 0.828301 | 2.104781 | 2.509529 |
| Day 1 | 2 | 1.683467 | 1.914954 | 3.608379 | 4.11433 |
| Day 3 | 3 | 2.485834 | 2.524736 | 5.022425 | 5.358388 |
| Day 7 | 4 | 3.533681 | 3.576595 | 6.352664 | 6.522491 |
| Day 10 | 5 | 4.531295 | 4.539544 | 7.498825 | 7.507845 |

TABLE 5
Percent Average Cumulative Drug released (ppm).

| | | % Average Cumulative Drug released (ppm) | | | |
|---|---|---|---|---|---|
| | | R and S | | V and W | |
| | Time | | Standard | | Standard |
| | point | Average | Deviation | Average | Deviation |
| Day 0 | 1 | 0.735090162 | 0.131819927 | 2.307155178 | 0.286199944 |
| Day 1 | 2 | 1.799210469 | 0.163685488 | 3.861354188 | 0.357761279 |
| Day 3 | 3 | 2.505284607 | 0.027507877 | 5.190406192 | 0.237561841 |
| Day 7 | 4 | 3.555137634 | 0.030344911 | 6.437577804 | 0.120085956 |
| Day 10 | 5 | 4.535419609 | 0.005832941 | 7.503335077 | 0.006377651 |

### Example 3—Redox-Responsive Nanoparticle System for Tyrosine Kinase Inhibitor Delivery to Treat Retinal Conditions

Purpose: A focus is developing drug delivery systems for prevention of vision loss and fibrosis. Reactive Oxygen Species (ROS) are implicated in many conditions, particularly in the eye. Age-related macular degeneration (AMD) and proliferative vitreoretinopathy (PVR) impact the retina. PVR is a vision-threatening complication after retinal detachment. PVR is caused by loss of cell-to-cell connection, elevated ROS, epithelial cell migration, and fibrotic membrane production. Previous work has demonstrated efficacy of tyrosine kinase inhibitor (TKI) Dasatinib (Das) in preventing PVR [1]. An objective was to develop an antioxidant-nanoparticle drug delivery system for sustained release of Das as a potential treatment for PVR to target ROS and fibrosis.

Methods: Antioxidant-nanoparticle delivery system as generated through oil-in-water self-assembly of 1,2-distearoyl-sn-glycero-3-phosphoethanolamine-poly(ethylene glycol)₃₄₀₀ (DSPE-PEG_{3.4}k) with a thiol containing polymer precursor based on published methods [2]. Das was dissolved in the oil phase before dropwise addition into ultrapure water under vigorous stirring. Particles were centrifuged any lyophilized. Nanoparticle size and zeta potential were determined by Brookhaven ZetaPals analyzer. Drug loading and release profile were determined by HPLC. In vitro release was determined in sink conditions in PBS, or PBS supplemented with 1 mM hydrogen peroxide. Cell viability was determined by MTS assay. Intracellular oxidative stress was measured by DCFDA assay.

A schematic diagram of particle preparation and oxidative stress for release is shown in FIG. 2.

Results: Thiol-based redox-responsive nanoparticles were successfully prepared with 227 nm average diameter (FIG. 4) with 0.3 PDI. Zeta potential of Das-loaded nanoparticles was measured as −29.2±1.0 mV (FIG. 3). The drug loading efficiency was 80% w/w. Release rates increased in response to oxidative stress (FIG. 1).

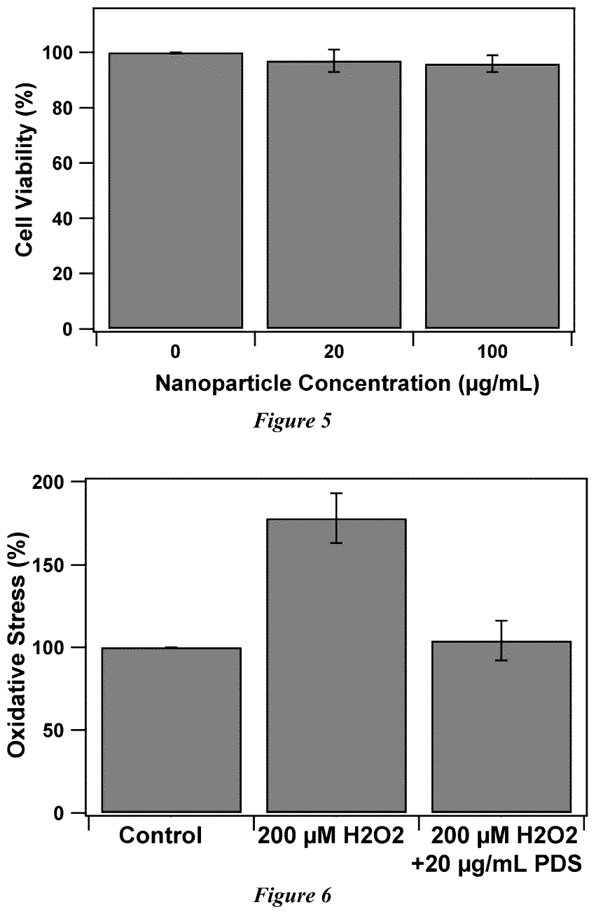

Antioxidant nanoparticles were nontoxic at concentrations below 100 μg/mL compared to control in ARPE-19 cells (FIG. 5). Cell viability was measured after 2 days.

Oxidative stress from 200 μM H₂O₂ was reduced by co-treatment of 20 μg/mL antioxidant-nanoparticle in ARPE-19 cells (FIG. 6). Oxidative stress was measured by DCFDA assay.

Conclusions: A thiol-based antioxidant nanoparticle system for redox-responsive release of a hydrophobic TKI was prepared and investigated. The TKI Dasatinib was loaded with high encapsulation efficiency. Release rates increased with exposure to oxidative stress. Ongoing studies include cellular uptake in primary retinal cells. Additional redox-responsive nanoparticle systems and therapeutics are under investigation for treatment of PVR.

### REFERENCES

[1] Ueda S et al. Graefes Arch Clin Exp Ophthalmol. 2021, 259 (4), 1103-1111
[2] Zhang R et al. Biomater Sci. 2023, 11 (12), 4254-4264.

### Example 4—Preparation and In Vitro Cytotoxicity of Redox-Responsive Nanoparticle System with and without Dasatinib Loading

Multiple batches of antioxidant-nanoparticles have been prepared as previously described through oil-in-water self-assembly of 1,2-distearoyl-sn-glycero-3-phosphoethanolamine-poly(ethylene glycol)3400 (DSPE-PEG3.4k) with a thiol containing polymer precursor. Parameters were modified to change particle size, such as sonication times and mixing speeds. Batches have been prepared unloaded and with Dasatinib incorporation. Size and zeta potential were characterized using dynamic light scattering (Malvern). Batch characterization is shown in Table 6.

A MTT assay [A1] was performed on these nanoparticles. Briefly, lyophilized nanoparticles were weighed to prepare solutions or suspensions of known concentrations in media containing 5% Fetal Bovine Serum 1% Antibiotic/Antimonic solution, and 94% Gibco DMEM+Glutamax. Particles were then homogenized using a sonicator for 30 seconds at 30% amplitude. Solutions were diluted to make the respective testing concentrations (10-1000 μg/mL) and warmed to 37° C.

Primary retinal pigment epithelial cells (RPE) cultured from porcine eyes obtained from an abattoir were seeded at a density of 25,000 cells per well in a 96 well plate. Cell containing well plates had their media aspirated and exchanged with 100 μL of the nanoparticle/media solutions and allowed to incubate for approximately 24 hours.

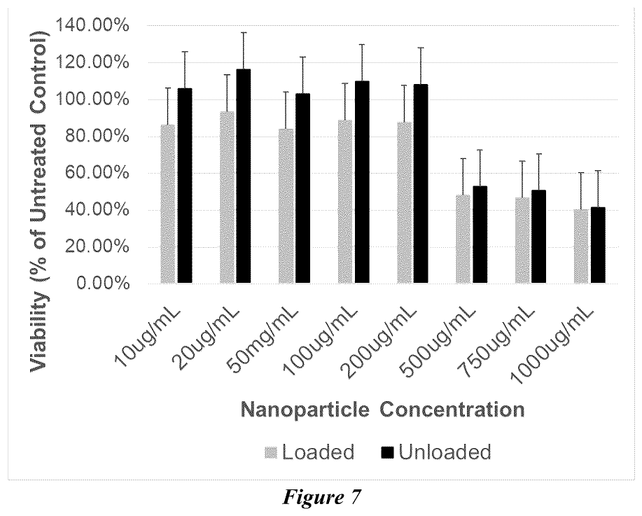

After 24 hours, medium was removed and replaced with 100 μL of fresh culture medium. Then 10 μL of the 12 mM MTT stock solution was added to each well. A negative control of 10 μL of the MTT stock solution added to 100 μL of medium alone was included. The plates were incubated for 4 hours at 37° C. All but 25 μL of media were removed from the wells. Then 50 μL of DMSO was added to each well and mixed thoroughly with a pipette. Plates were incubated for 10 min at 37° C. Samples were mixed again and absorbance read at 540 nm. Results are shown in FIG. 7, demonstrating high cell viability at 200 μg/mL with a decrease at 500 μg/mL. Dasatinib loading decreased cell viability compared to unloaded particles.

TABLE 6
Additional Batch Sizing, Polydispersity
Index (PDI), and Zeta Potential.

| | | | | Zeta | | | |
|---|---|---|---|---|---|---|---|
| | | Size (nm) | | Potential (mV) | | PDI | |
| | Load- | Aver- | Standard | Aver- | Standard | Aver- | Standard |
| Batch | ed | age | Deviation | age | Deviation | age | Deviation |
| 1 | Yes | 669.9 | 176.9 | −25.5 | 0.8 | 0.63 | 0.10 |
| 2 | No | 89.1 | 1.6 | −26.1 | 1.4 | 0.29 | 0.02 |
| 3a | No | 6433.6 | 668.1 | * | * | 0.52 | 0.25 |
| 3b | Yes | 304.3 | 60.5 | * | * | 0.41 | 0.06 |
| 4 | Yes | 1201.8 | 767.3 | −26.4 | 2.6 | 0.84 | 0.23 |

* not measured

### REFERENCES

[A1] Kumar P, Nagarajan A, Uchil P D. Analysis of Cell Viability by the MTT Assay. Cold Spring Harb Protoc. 2018 Jun. 1;2018 (6). doi: 10.1101/pdb.prot095505. PMID: 29858338.

### Example 5

Retinal detachment (RD) is a serious condition that requires timely intervention as it will lead to blindness if left unattended. Corrective surgery for RD involves vitrectomy and reattachment of the retina. The vitreous cannot be transplanted or regenerated, so it must be replaced with a substitute following vitrectomy. Currently used substitutes for tamponade include air/gas, silicone oil, or perfluorocarbons, but are known to have significant limitations and complications. Silicone oil is widely used as a post-vitrectomy tamponade in complicated RD cases, but its emulsification can cause various issues. Furthermore, due to the lighter-than-water nature of conventional silicone oil, it requires an uncomfortable face-down prone position on patients with inferior retinal tears/holes. Such prone position is impossible for infants, many pediatric patients, or other complex patient populations to maintain. Failure of the patient to maintain the required head position increases the risk of retina re-detachment and/or proliferative vitreoretinopathy (PVR), the major complication leading to retinal reattachment failure. While perfluorocarbons can be effective tamponades for eyes with inferior retinal tears/holes owing to their heavier-than-water specific gravity, they can only be used for a relatively short time due to known ocular toxicity. As PVR often takes up to 3 months to develop, perfluorocarbons need to be replaced with another tamponade such as silicone oil. In contrast to currently used hydrophobic tamponades, which utilize surface tension and buoyancy to tamponade the retina, the vitreous body exerts its tamponade effect via viscoelasticity and swelling pressure, both of which can be mimicked by hydrogels. Prior research has demonstrated great potential to use hydrogels as vitreous substitutes, and short-term hyaluronan-based substitutes are now in Phase I clinical trials (e.g., Vitargus®).

PVR remains the major complication following retinal reattachment surgery, affecting 5-10% of rhegmatogenous RD (RRD) patients, despite improvement in surgical skills and postsurgical treatment methods over the years, with a long list of conditions increasing incidence of PVR. Retinal detachment involving ocular trauma such as penetrative injury has an even higher rate of PVR, affecting up to 60% of eyes. While retinal reattachment (anatomical success) can eventually be achieved in PVR patients following several surgeries, visual acuity (function) is often significantly diminished, with the worst cases resulting in blindness. Thus, 1 out of 10-20 patients undergoing RRD repair, and up to 1 out of 2 patients for surgical repair of RD associated with posterior ocular trauma will have significantly diminished vision. In order to reduce the incidence of PVR, adjunctive treatment using pharmacological agents has been suggested. Various studies have identified multiple promising therapeutic targets and/or agents/drugs, using in vitro cell models and/or in vivo animal models. Unfortunately, there has yet to be a treatment approved for prevention and/or treatment of PVR as candidate drugs successful in experimental systems have failed in clinical trials. One potential reason for lack of success in clinical trials is failure to maintain intravitreal concentration to achieve therapeutic effect. Failed trials only utilized a limited number of intravitreal injections (often a single injection) for drug delivery that cannot maintain effective dosing. The recent GUARD trial using methotrexate has tried to overcome this issue via multiple intravitreal injections (13 times over 16 weeks). Such a sheer number of required injections may reduce patient compliance and adherence to the treatment even if successful in preventing PVR, and vitreoretinal surgeons are already overburdened with frequent injections for other retinal conditions. Thus, a sustained drug delivery system that can maintain effective drug dose without many intravitreal injections needs to be delivered for clinical use, improving patient compliance, visual outcomes, and reducing health care costs.

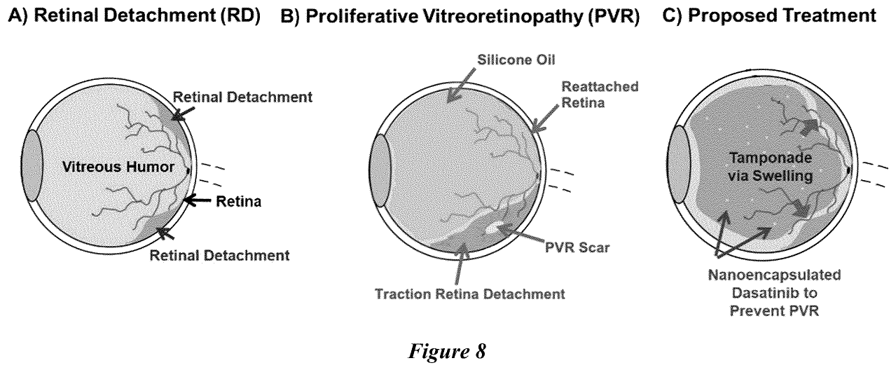

Scientific Challenge: Currently, there are no treatment methods that are capable of locally releasing therapeutics to prevent complications like PVR. The scientific challenge is protecting the delivered drug dasatinib from degradation and sustaining its local release. To address this challenge, an injectable vitreous substitute that serves as a drug delivery reservoir to enable localized and sustained delivery of dasatinib inside the eye is described herein (FIG. 8). By delivering therapeutics directly to the retina, ocular health and function can be dramatically improved following vitrectomy.

Clinical Challenge: Over 250,000 vitrectomies are performed annually in the US with direct costs of $1.9B. Global incidence of vitrectomy has increased 70% in the past 10 years, and is expected to continue to rise due to population demographics and use of vitrectomy to treat floaters. Quality of life is significantly decreased by visual impairment, primarily affecting reading and driving. Further, pediatric retinopathy of prematurity (ROP) patients are commonly treated with vitrectomy, and disproportionately suffer from poor visual outcomes. With the number of vitrectomies performed on the rise, a projected shortage of ophthalmologists, and recent evidence indicating that vitrectomy directly contributes to several causes of blindness including PVR, this is an important and timely topic. Furthermore, modulating retinal reattachment with an improved vitreous substitute that can locally deliver therapeutics and potentially prevent fibrosis and complications after surgery will improve patient visual outcomes.

Herein, a nanodelivery system is tested to sustain release of the drug dasatinib, which we have shown to prevent PVR associated changes both in vitro and in vivo. While the focus of this research is on the prevention and treatment of fibrosis after vitrectomy, an injectable system capable of protecting and delivering anti-fibrotic therapeutics and tyrosine kinase inhibitors (e.g., dasatinib) has potential in other conditions, including outside the eye. The work herein can generate scientific knowledge on the impact of localized drug delivery for prevention or treatment of fibrosis.

Innovation. Nanoencapsulated therapeutics to prevent PVR in high risk eyes is innovative.

The use of biodegradable, redox-responsive, and reactive oxygen species (ROS) scavenging polydopamine (PDA) nanoparticles (NPs) and polydisulfide (PDS) NPs to extend release of dasatinib are described herein. An aim is to prevent PVR, which usually occurs within three months of vitrectomy. Using a delivery system that degrades in vivo is suitable as it does not require removal. Furthermore, the redox-responsive nature of the particles will release more drug upon inflammation, a key process of PVR development. These NPs have yet to be evaluated in a nanocomposite or for drug delivery of tyrosine kinase inhibitors.

The results have the potential to not only change the technology used for PVR treatment, but for a paradigm shift in ophthalmic disease treatment—focusing on prevention—that involves vitrectomy.

### Approach

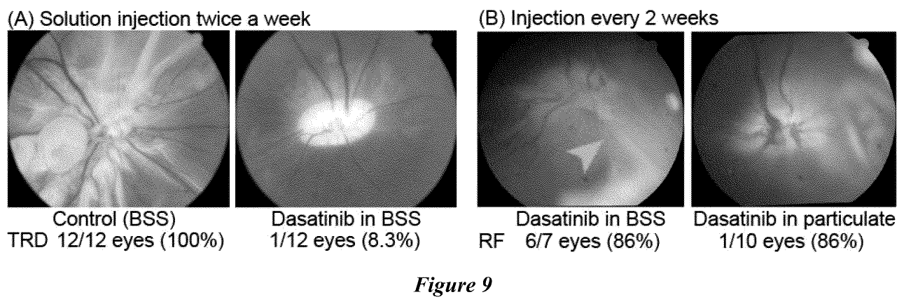

Preliminary Data. Building on preliminary data, it is proposed to nanoencapsulate dasatinib to sustain release. It has previously been demonstrated that dasatinib prevents PVR associated changes in pig models (FIG. 9). Multiple intravitreal injections (twice a week) of dasatinib solution prevented traction retinal detachment (TRD), while a sustained delivery of dasatinib incorporated into biodegradable poly(lactic-co-glycolic acid) (PLGA) particles prevented formation of full thickness retinal fold in a traumatic PVR model. Thus, these results clearly show that sustained presence of dasatinib can prevent PVR associated changes in vivo. However, due to potential acidification upon PLGA breakdown resulting in ocular inflammation induced by PLGA in some studies, as well as the short sustained release profile (2 weeks) and aggregation of our PLGA particles, new nanoparticle delivery systems are evaluated herein. All in vivo safety and efficacy studies can be conducted in pig models.

Optimizing nanoparticle composition. It is hypothesized that an effective tamponade will not be sufficient on its own, especially with eyes that have high risk for developing PVR.

PVR remains the major complication of retinal reattachment surgery, and is currently without any treatment for prevention. Proof of concept studies have clearly shown that adjunctive treatment can be beneficial for PVR prevention, but clinical trials have been unsuccessful, in part, due to lack of effective method for sustained delivery of potentially useful drugs. Dasatinib (Das), a tyrosine kinase inhibitor, will be evaluated because it has previously been demonstrated to prevent PVR in porcine models. An aim is to develop nanosystems for sustained delivery.

### Nanoparticle Synthesis and Characterization

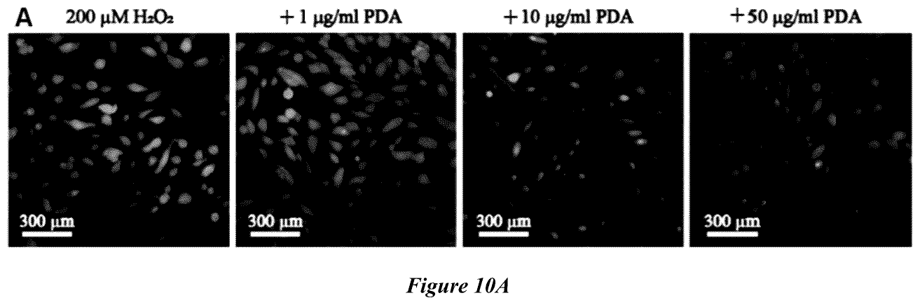

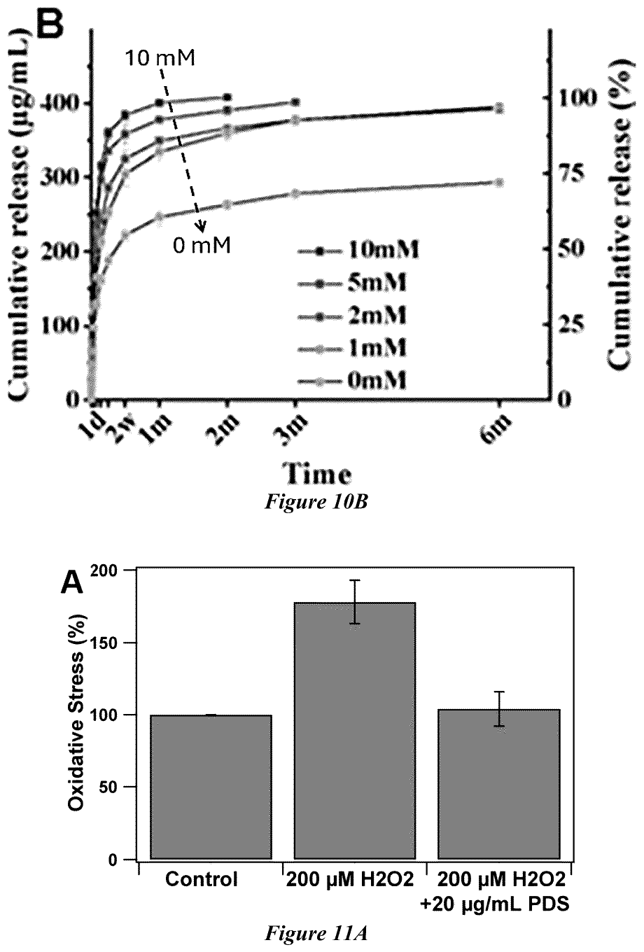

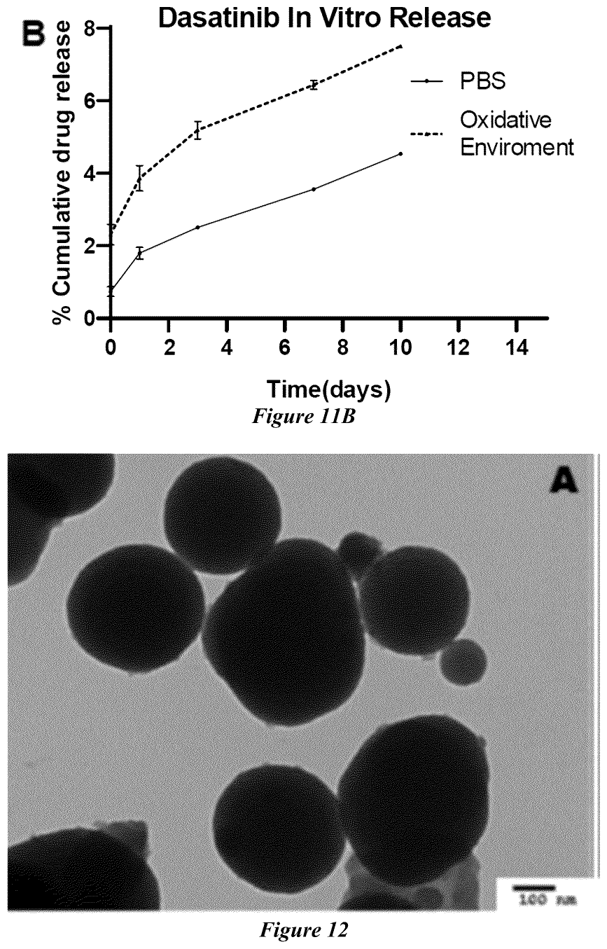

Dasatinib Nanoencapsulation: The encapsulation strategy is intended to facilitate rapid initial release of the therapeutic, which will be desirable for immediate protection of the retina after vitrectomy, followed by controlled release to maintain therapeutic concentration for at least 1 month. Das has been loaded into preliminary NP formulations with different benefits: PDA NPs and PDS NPs. PDA NPs were selected for further investigation in this study due to their ability to scavenge ROS (FIG. 10A), which has been implicated in PVR, and sustain release for over a month (FIG. 10B). PDS NPs also scavenge ROS (FIG. 11A) and have demonstrated potential to sustain release of dasatinib (FIG. 11B).
PDA NPs will be synthesized and loaded with dasatinib using published techniques. Briefly, dopamine hydrochloride will be added to DI H₂O containing NaOH and stirred at 500 rpm for 3 h. For therapeutic loaded NPs, dasatinib will then be added and stirred another 21 hours. Therapeutic loading targets will be up to 30 mM based on preliminary data. In pilot studies, dasatinib was loaded on PDA NPs at 0.1 mM concentration.
PDS NPs will be synthesized and loaded with dasatinib based on modifications of a published protocol. Briefly, this system is generated through oil-in-water self-assembly of 1,2-distearoyl-sn-glycero-3-phosphoethanolamine-poly(ethylene glycol) 3400 (DSPE-PEG3.4k) with a thiol containing polymer precursor based on published methods. Das will be dissolved in the thiol-based precursor before dropwise addition into ultrapure water at 1 mL/min under vigorous stirring for 12 hours. Particles will be centrifuged and lyophilized.

We have demonstrated preliminary loading of dasatinib into the PDA NPs and PDS NPs, as evidenced by increase in both size and zeta potential after loading (Table 7).

TABLE 7
Size, PDI, and zeta potential of pilot PDA NPs and
PDS NPs with and without dasatinib (Das) loading.

| | PDA NPs | Das-PDA NPs | PDS NPs | Das-PDS NPs |
|---|---|---|---|---|
| Size (nm) | 162 | 182 | 89 | 266 |
| PDI | 0.005 | 0.01 | 0.29 | 0.41 |
| Zeta potential (mV) | −38.17 ± 0.90 | −31.58 ± 0.96 | −26.11 ± 1.38 | −25.48 ± 0.82 |

Characterization: NP morphology will be evaluated with transmission electron microscopy (TEM) (FIG. 12), and diameter will be determined by measuring at least 100 NPs from TEM images using ImageJ (NIH). A Zetasizer (Malvern) will be used to determine hydrodynamic diameter and polydispersity (PD), and ZetaPals (Malvern) will be used to determine zeta potential before and after therapeutic loading.

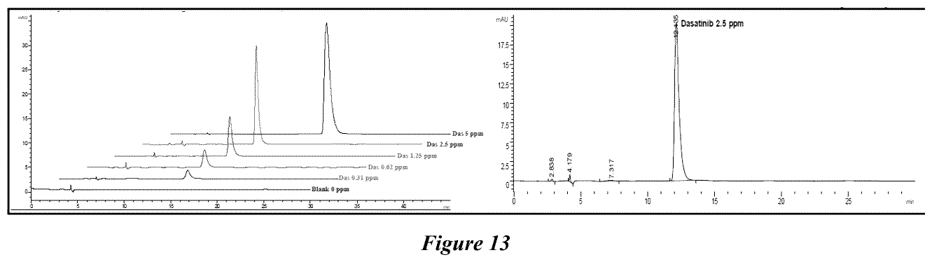

Dasatinib Loading and Release: Loading in PDA and PDS NPs will be determined by dissolving therapeutic-loaded particles in H₂O₂. Free unencapsulated Das will precipitate with centrifugation and water washes and will be extracted with methanol for quantification. Therapeutic will then be measured by high performance liquid chromatography (HPLC), and percent therapeutic relative to mass of NPs will be calculated. Release rates of dasatinib from the particles will be evaluated by incubating particles (blank and loaded, with and without hydrogel) in 4 mL PBS (to simulate the volume of the vitreous humor) at 37° C. with shaking. Tween 80 (0.1%) can be added to the release media to overcome saturation issues associated with hydrophobic Das. Eluent will be removed and replaced after 1, 6, 12, and 24 hours, then on days 3, 7, 14, 21, and 28. Studies will be continued beyond 28 days as needed until all therapeutic is released. Eluent samples will also be quantified using a developed HPLC method with 50:50 methanol: H₂O mobile phase at 323 nm absorbance (FIG. 13). Diffusion coefficients will be calculated using our published methods and release data will be fitted to standard kinetic models as we have done in previous studies. In our preliminary studies, the PDS NPs had an average Das encapsulation efficiency of 80+5%.

### In Vitro Characterization Using RPE Cells and Ex Vivo Retina Tissue.

Primary cultured porcine RPE and MG cells as well as human iCell RPE cells (Fujifilm Cellular Dynamics, Inc.) will be used to assess the effect of dasatinib-loaded NPs in vitro. Most of the studies will be conducted with porcine cells, and iCell RPE and primary human RPE and MG will be utilized to confirm that key findings obtained with porcine cells are reproduced in human cells. NPs will be prevented from directly contacting cells by adding NPs to the transwell upper chamber while cells are cultured at the bottom of the plate as previously described. The effect of dasatinib released from NPs on cellular fibrotic changes, including epithelial-to-mesenchymal transition (EMT) as well as proliferation and matrix contraction of RPE/MG cells, will be examined using published methods, but modified for 24 well plates. Apoptosis will be examined by TUNEL staining. Particles without dasatinib loading, unencapsulated dasatinib, and dasatinib loaded PLGA NPs will serve as controls.

### Test for Adverse Effect of Nanoparticles In Vivo in a Pig Model

A porcine model will be used to determine if any adverse effects are induced by nanoparticles.

Prior to surgery, ocular examination utilizing a tonometer, slit lamp and indirect ophthalmoscopes, and OCT will be conducted to check for ocular abnormalities. Baseline ERG measurement will also be performed prior to nanoparticle injection.

Animals will then be injected in one eye with a nanoparticle suspension in BSS, while the fellow eye will be injected with BSS (vehicle alone) to serve as a control. Vision is not significantly affected by vehicle injection, and thus, the use of both eyes is justified.

On post-injection days 3, 14, and 28, eyes will be examined using a tonometer, OCT, slit lamp and indirect ophthalmoscopes to check for inflammation and any changes to IOP, cornea, lens, or retina. ERG measurement will also be conducted on day 28. The effect of nanoparticles on retinal function will be examined in the same animal by measuring ERG b-wave amplitude prior to (baseline) and after nanoparticle treatment (day 28) as well as comparing the two eyes in the same animal with and without the nanoparticles. A 50% reduction in b-wave amplitude will be defined as an adverse effect to retinal function. Using a paired t-test, with sample size of n=10 animals, this study has 80% power to detect a difference in 50% reduction of b-wave amplitude after nanoparticle treatment. Tests are two-sided and type one error is set at 0.05. R version 2.9.2 was used for sample size calculation. Considering a waste rate of 15-20%, it is estimates that n=12 animals are required for each of the test groups. Equal numbers of both sexes will be used.

Following ERG measurements and ocular examination on day 28, animals will be euthanized and eyes enucleated. The anterior segment will be removed from eyes following mild fixation, and the vitreous with dasatinib-loaded nanoparticles will be collected, weighed, and assessed for remaining content of dasatinib using HPLC. The remaining eye cup will be processed for histology to examine retinal structures, apoptosis (TUNEL staining), Müller glia (MG) activation (GFAP immunostaining), and microglia and/or infiltrating immune cell localization (Iba-1 immunostaining). These histological analyses will be integrated with ERG data analyses to assess the presence or absence of adverse effects of the NP formulations on the retina.

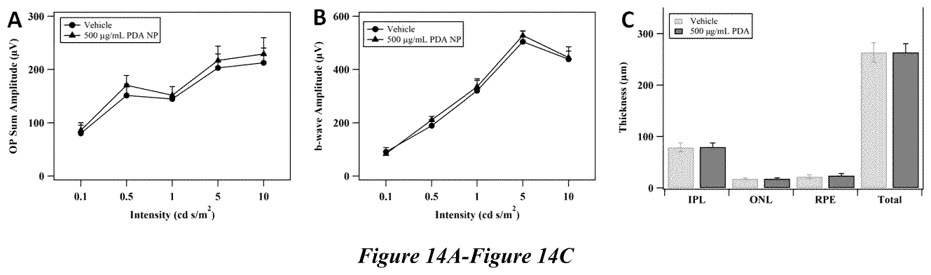

Outcomes/Benchmarks for Success: Benchmarks include successful loading to enable release of dasatinib at an effective dose (0.03-0.3 μM). This is the target concentration in the media or vitreous following release. An initial target is loading dasatinib into the nanoparticles at higher levels (30 μM) to account for sustained release and the short half-life of the drug in the vitreous (1-2 hours). A target NP size will be <300 nm to prevent visual impairment while having appropriate loading and release profiles to provide initial dasatinib concentrations of 0.3 μM and sustain release for at least 4 weeks. RPE and MG viability should not be significantly different from the negative control, and is not predicted based on preliminary studies. Dasatinib prevents PVR associated changes of RPE and MG cells, and it is predicted that dasatinib-loaded NPs can prevent fibrotic cellular changes in vitro, as determined by quantifying markers of EMT: proliferation, matrix contraction, and fibronectin and collagen expression. Quantifiable measures to evaluate for statistical significance compared to vehicle control in vivo include clinical examination scores, ERG, and histology of retinal sections. Significant adverse effects of dasatinib-loaded NPs are not projected, as neither dasatinib nor PDA NPs have shown detectable change in ERG response. PDA NP toxicity is de-risked by evaluating after intravitreal injection in chicks (FIG. 14A-FIG. 14C) and mice. This is the first evaluation of PDS NPs after intravitreal injection, but toxicity issues are not projected based on favorable in vivo outcomes of these components in other delivery applications.

Potential Alternative Approaches: A potential problem includes short-term release. The risk of excessive burst release or short-term release has been minimized by evaluating multiple NP compositions, with demonstrated extended release potential from PDA and PDS NPs. The therapeutic loading target levels of 30 μM and NP concentration targets for injection of 50 μg/mL can be modified if either in vitro or in vivo screening demonstrates significant burst release, limited efficacy, or adverse effects at these concentrations.

Sex as a Biological Variable: There are no known sex differences in etiology of PVR after adjusting for clinical status of the eye. No difference in cellular function including proliferation and myofibroblast transdifferentiation based on sex has been observed. While there is no report exclusively showing the effect of sex difference on PVR development, both sexes will be utilized for experiments. Sex will be noted, and statistical analyses will be conducted to examine if sex acted as a factor involved in PVR development.

Rigor, Reproducibility, and Transparency: To increase rigor, several correlating methods and outcomes measures will be used. For reproducibility, all experiments will be performed a minimum of three times. Statistical analysis will be performed on all quantitative data using R (version 4.2.1). Normality of data will be assessed using the Shapiro-Wilk test. Data transformation will be performed if necessary or nonparametric methods (Mann-Whitney U Test or Kruskal-Wallis test) will be used. ANOVA models or linear mixed-effects models will be used for analysis. Holm's method will be used for adjusting for multiple comparisons between treatment conditions. Sample size calculations were based on preliminary data or similar published results. For in vitro cell and tissue culture experiments, n=7 replicates will be used for each condition to have 80% power for a 2-fold difference between conditions with α=0.01 and coefficients of variance (CV)-30%. Power analysis and justification for each in vivo study was described above. For transparency, all experimental details will be disclosed in publications and raw data will be deposited in public databases. To promote unbiased design, all experiments were designed with positive and negative controls. Where manual measurements are made, at least two individuals will perform these analyses independently. Well plates and histology slides will be coded in a way that masks investigators from knowing the condition.

Impact: This project completion will result in compositions with the ability to locally sustain release therapeutics to prevent complications after vitrectomy. In future studies, other therapeutics can be explored for controlled release, particularly in disease models of ROP, diabetic retinopathy, retinoblastoma, and uveal melanoma. The developed technologies also have potential to treat other diseases outside the eye, particularly cancers (e.g., leukemia) which are being treated with dasatinib.

### EXEMPLARY ASPECTS

In view of the described compositions, devices, systems, and methods, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: A pharmaceutical composition comprising: a plurality of redox-responsive disulfide nanoparticles; and a tyrosine kinase inhibitor encapsulated within each of the redox-responsive disulfide nanoparticles.

Example 2: The pharmaceutical composition of any example herein, particularly example 1, wherein the redox-responsive disulfide nanoparticles are derived from a phospholipid-PEG.

Example 3: The pharmaceutical composition of any example herein, particularly examples 1-2, wherein the redox-responsive disulfide nanoparticles are derived from diacylphospholipid-poly(ethylene glycol).

Example 4: The pharmaceutical composition of any example herein, particularly examples 1-3, wherein the redox-responsive disulfide nanoparticles are derived from Thiol 1,2-Dioleoyl-sn-glycero-3-phosphoethanolamine (DOPE)-polyethylene glycol (PEG).

Example 5: The pharmaceutical composition of any example herein, particularly examples 1-4, wherein the redox-responsive disulfide nanoparticles are derived from 1,2-distearoyl-sn-glycero-3-phosphoethanolamine-poly(ethylene glycol) and a thiol containing polymer precursor.

Example 6: The pharmaceutical composition of any example herein, particularly example 5, wherein the thiol containing polymer precursor comprises 1,4-butane diol bis(thioglycolate).

Example 7: The pharmaceutical composition of any example herein, particularly examples 1-5, wherein the redox-responsive disulfide nanoparticles are substantially spherical in shape.

Example 8: The pharmaceutical composition of any example herein, particularly examples 1-7, wherein the redox-responsive disulfide nanoparticles have an average particle size of from 30 nanometers (nm) to 10 micrometers (microns, μm).

Example 9: The pharmaceutical composition of any example herein, particularly examples 1-8, wherein the redox-responsive disulfide nanoparticles have an average particle size of from 30 nm to 950 nm.

Example 10: The pharmaceutical composition of any example herein, particularly examples 1-9, wherein the redox-responsive disulfide nanoparticles have an average particle size of from 90 nm to 7 μm.

Example 11: The pharmaceutical composition of any example herein, particularly examples 1-10, wherein the redox-responsive disulfide nanoparticles have an average particle size of from 100 nanometers (nm) to 500 nanometers.

Example 12: The pharmaceutical composition of any example herein, particularly examples 1-11, wherein the redox-responsive disulfide nanoparticles have an average particle size of from 200 to 250 nanometers, such as 227 nm.

Example 13: The pharmaceutical composition of any example herein, particularly examples 1-12, wherein the redox-responsive disulfide nanoparticles have a polydispersity index of 1.0 or less.

Example 14: The pharmaceutical composition of any example herein, particularly examples 1-13, wherein the redox-responsive disulfide nanoparticles have a polydispersity index of 0.85 or less.

Example 15: The pharmaceutical composition of any example herein, particularly examples 1-14, wherein the redox-responsive disulfide nanoparticles have a polydispersity index of 0.5 or less.

Example 16: The pharmaceutical composition of any example herein, particularly examples 1-15, wherein the redox-responsive disulfide nanoparticles have a polydispersity index of 0.3 or less.

Example 17: The pharmaceutical composition of any example herein, particularly examples 1-16, wherein the tyrosine kinase inhibitor is hydrophobic.

Example 18: The pharmaceutical composition of any example herein, particularly examples 1-17, wherein the tyrosine kinase inhibitor comprises dasatinib.

Example 19: The pharmaceutical composition of any example herein, particularly examples 1-18, wherein the tyrosine kinase inhibitor is encapsulated within the redox-responsive disulfide nanoparticle with an encapsulation efficiency of 50% or more, such as 80% or more.

Example 20: The pharmaceutical composition of any example herein, particularly examples 1-19, wherein the redox-responsive disulfide nanoparticles encapsulating the tyrosine kinase inhibitor has an average Zeta potential of from −20 to −40 mV.

Example 21: The pharmaceutical composition of any example herein, particularly examples 1-20, wherein the redox-responsive disulfide nanoparticles encapsulating the tyrosine kinase inhibitor can have an average Zeta potential of from −25 to −35 mV.

Example 22: The pharmaceutical composition of any example herein, particularly examples 1-21, wherein the redox-responsive disulfide nanoparticles encapsulating the tyrosine kinase inhibitor can have an average Zeta potential of from −25 to −30 mV.

Example 23: The pharmaceutical composition of any example herein, particularly examples 1-22, wherein the redox-responsive disulfide nanoparticles encapsulating the tyrosine kinase inhibitor can have an average Zeta potential of −29.2±1.0 mV.

Example 24: The pharmaceutical composition of any example herein, particularly examples 1-22, wherein the redox-responsive disulfide nanoparticles encapsulating the tyrosine kinase inhibitor can have an average Zeta potential of −25.5±1.0 mV.

Example 25: The pharmaceutical composition of any example herein, particularly examples 1-24, wherein the pharmaceutical composition releases the tyrosine kinase inhibitor in response to a redox condition, for example when subjected to oxidative stress and/or in the presence of reactive oxygen species.

Example 26: The pharmaceutical composition of any example herein, particularly examples 1-25, wherein the pharmaceutical composition is substantially non-toxic at concentration of 500 μg/mL or less.

Example 27: The pharmaceutical composition of any example herein, particularly examples 1-26, wherein the pharmaceutical composition is substantially non-toxic at concentration of 200 μg/mL or less.

Example 28: The pharmaceutical composition of any example herein, particularly examples 1-27, wherein the pharmaceutical composition is substantially non-toxic at concentration of 100 μg/mL or less.

Example 29: The pharmaceutical composition of any example herein, particularly examples 1-28, wherein the pharmaceutical composition further comprises a solvent, a carrier, an excipient, an additional therapeutic agent (optionally encapsulated within the redox-responsive disulfide nanoparticles), or a combination thereof.

Example 30: A method of making the pharmaceutical composition of any example herein, particularly examples 1-29.

Example 31: The method of any example herein, particularly example 30, wherein the method comprises oil-in-water self-assembly.

Example 32: A method of treating or preventing an ocular injury, disease, or disorder in a subject in need thereof, the method comprising administering a therapeutically effective amount of a plurality of redox-responsive disulfide nanoparticles to the subject.

Example 33: The method of any example herein, particularly example 32, wherein the redox-responsive disulfide nanoparticles are derived from a phospholipid-PEG.

Example 34: The method of any example herein, particularly examples 32-33, wherein the redox-responsive disulfide nanoparticles are derived from diacylphospholipid-poly(ethylene glycol).

Example 35: The method of any example herein, particularly examples 32-34, wherein the redox-responsive disulfide nanoparticles are derived from Thiol 1,2-Dioleoyl-sn-glycero-3-phosphoethanolamine (DOPE)-polyethylene glycol (PEG).

Example 36: The method of any example herein, particularly examples 32-35, wherein the redox-responsive disulfide nanoparticles are derived from 1,2-distearoyl-sn-glycero-3-phosphoethanolamine-poly(ethylene glycol) and a thiol containing polymer precursor.

Example 37: The method of any example herein, particularly example 36, wherein the thiol containing polymer precursor comprises 1,4-butane diol bis(thioglycolate).

Example 38: The method of any example herein, particularly examples 32-37, wherein the redox-responsive disulfide nanoparticles are substantially spherical in shape.

Example 39: The method of any example herein, particularly examples 32-38, wherein the redox-responsive disulfide nanoparticles have an average particle size of from 30 nanometers (nm) to 10 micrometers (microns, μm).

Example 40: The method of any example herein, particularly examples 32-39, wherein the redox-responsive disulfide nanoparticles have an average particle size of from 30 nm to 950 nm.

Example 41: The method of any example herein, particularly examples 32-40, wherein the redox-responsive disulfide nanoparticles have an average particle size of from 90 nm to 7 μm.

Example 42: The method of any example herein, particularly examples 32-41, wherein the redox-responsive disulfide nanoparticles have an average particle size of from 100 nanometers (nm) to 500 nanometers.

Example 43: The method of any example herein, particularly examples 32-42, wherein the redox-responsive disulfide nanoparticles have an average particle size of from 200 to 250 nanometers, such as 227 nm.

Example 44: The method of any example herein, particularly examples 32-43, wherein the redox-responsive disulfide nanoparticles have a polydispersity index of 1.0 or less

Example 45: The method of any example herein, particularly examples 32-44, wherein the redox-responsive disulfide nanoparticles have a polydispersity index of 0.85 or less.

Example 46: The method of any example herein, particularly examples 32-45, wherein the redox-responsive disulfide nanoparticles have a polydispersity index of 0.5 or less.

Example 47: The method of any example herein, particularly examples 32-46, wherein the redox-responsive disulfide nanoparticles have a polydispersity index of such as 0.3 or less.

Example 48: The method of any example herein, particularly examples 32-47, wherein a therapeutic agent is encapsulated within each the redox-responsive disulfide nanoparticles.

Example 49: The method of any example herein, particularly example 48, wherein the therapeutic agent comprises an anticancer agent, an anti-inflammatory agent, an antimicrobial agent, an anti-oxidant agent, an antibody, a protein, or a combination thereof.

Example 50: The method of any example herein, particularly examples 48-49, wherein the therapeutic agent comprises a chemotherapeutic agent, an immunotherapeutic agent, or a combination thereof.

Example 51: The method of any example herein, particularly examples 48-50, wherein the therapeutic agent is hydrophobic.

Example 52: The method of any example herein, particularly examples 48-51, wherein the therapeutic agent is a tyrosine kinase inhibitor.

Example 53: The method of any example herein, particularly example 52, wherein the tyrosine kinase inhibitor comprises dasatinib.

Example 54: The method of any example herein, particularly examples 48-53, wherein the therapeutic agent is encapsulated within the redox-responsive disulfide nanoparticles with an encapsulation efficiency of 50% or more, such as 80% or more.

Example 55: The method of any example herein, particularly examples 48-54, wherein the redox-responsive disulfide nanoparticles release the therapeutic agent in response to a redox condition, for example when subjected to oxidative stress and/or in the presence of reactive oxygen species.

Example 56: A method of treating or preventing an ocular injury, disease, or disorder in a subject in need thereof, the method comprising administering a therapeutically effective amount of the pharmaceutical composition of any example herein, particularly examples 1-29 to the subject.

Example 57: The method of any example herein, particularly example 56, wherein the pharmaceutical composition releases the tyrosine kinase inhibitor in response to a redox condition, for example when subjected to oxidative stress and/or in the presence of reactive oxygen species.

Example 58: The method of any example herein, particularly examples 32-57, wherein the ocular injury, disease, or disorder generates oxidative stress.

Example 59: The method of any example herein, particularly examples 32-58, wherein the ocular injury, disease, or disorder generates reactive oxygen species (ROS).

Example 60: The method of any example herein, particularly examples 32-59, wherein the ocular injury comprises injury to the cornea and/or ocular nerve.

Example 61: The method of any example herein, particularly examples 32-60, wherein the ocular disease or disorder comprises a degenerative disease or disorder.

Example 62: The method of any example herein, particularly examples 32-61, wherein the ocular disease or disorder comprises a retinal condition.

Example 63: The method of any example herein, particularly examples 32-62, wherein the ocular disease or disorder comprises age-related macular degeneration, proliferative vitreoretinopathy (PVR), diabetic retinopathy, glaucoma, retinitis pigmentosa, inherited retinal diseases, retinal tears/holes/detachments, or a combination thereof.

Example 64: The method of any example herein, particularly examples 32-63, wherein the ocular disease or disorder comprises age-related macular degeneration, proliferative vitreoretinopathy (PVR), or a combination thereof.

Example 65: The method of any example herein, particularly examples 32-64, wherein the ocular disease or disorder comprises proliferative vitreoretinopathy (PVR).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

What is claimed is:

1. A pharmaceutical composition comprising:
a plurality of redox-responsive disulfide nanoparticles; and
a tyrosine kinase inhibitor encapsulated within each of the redox-responsive disulfide nanoparticles.

2. The pharmaceutical composition of claim 1, wherein the redox-responsive disulfide nanoparticles are derived from a phospholipid-PEG.

3. The pharmaceutical composition of claim 1, wherein the redox-responsive disulfide nanoparticles are derived from 1,2-distearoyl-sn-glycero-3-phosphoethanolamine-poly(ethylene glycol) and a thiol containing polymer precursor.

4. The pharmaceutical composition of claim 3, wherein the thiol containing polymer precursor comprises 1,4-butane diol bis(thioglycolate).

5. The pharmaceutical composition of claim 1, wherein the redox-responsive disulfide nanoparticles are substantially spherical in shape.

6. The pharmaceutical composition of claim 1, wherein the redox-responsive disulfide nanoparticles have an average particle size of from 30 nanometers (nm) to 10 micrometers (microns, μm).

7. The pharmaceutical composition of claim 1, wherein the redox-responsive disulfide nanoparticles have an average particle size of from 100 nanometers (nm) to 500 nanometers.

8. The pharmaceutical composition of claim 1, wherein the redox-responsive disulfide nanoparticles have a polydispersity index of 0.85 or less.

9. The pharmaceutical composition of claim 1, wherein the tyrosine kinase inhibitor is hydrophobic.

10. The pharmaceutical composition of claim 1, wherein the tyrosine kinase inhibitor comprises dasatinib.

11. The pharmaceutical composition of claim 1, wherein the tyrosine kinase inhibitor is encapsulated within the redox-responsive disulfide nanoparticle with an encapsulation efficiency of 50% or more.

12. The pharmaceutical composition of claim 1, wherein the redox-responsive disulfide nanoparticles encapsulating the tyrosine kinase inhibitor has an average Zeta potential of from −20 to −40 mV.

13. The pharmaceutical composition of claim 1, wherein the pharmaceutical composition is substantially non-toxic at concentration of 200 μg/mL or less.

14. The pharmaceutical composition of claim 1, wherein the pharmaceutical composition further comprises a solvent, a carrier, an excipient, an additional therapeutic agent, or a combination thereof.

15. A method of making the pharmaceutical composition of claim 1, wherein the method comprises oil-in-water self-assembly.

16. A method of treating or preventing an ocular injury, disease, or disorder in a subject in need thereof, the method comprising administering a therapeutically effective amount of the pharmaceutical composition of claim 1 to the subject.

17. The method of claim 16, wherein the pharmaceutical composition releases the tyrosine kinase inhibitor in response to a redox condition.

18. The method of claim 17, wherein the pharmaceutical composition releases the tyrosine kinase inhibitor when subjected to oxidative stress and/or in the presence of reactive oxygen species.

19. The method of claim 16, wherein the ocular disease or disorder comprises proliferative vitreoretinopathy (PVR).

20. A method of treating or preventing an ocular injury, disease, or disorder in a subject in need thereof, the method comprising administering a therapeutically effective amount of a plurality of redox-responsive disulfide nanoparticles to the subject.
